(19) United States
(12) Patent Application Publication
(10) Pub. No.: US 2026/0315776 A1
(43) Pub. Date: Oct. 15, 2026

(54) BLOOD PUMP AND BLOOD PUMP SYSTEM

(71) Applicant: ABIOMED Europe GmbH, Aachen (DE)

(72) Inventors: Frank Kirchhoff, Aachen (DE); Thorsten Siess, Aachen (DE); Wolfgang Kerkhoffs, Aachen (DE)

(21) Appl. No.: 19/473,820

(22) PCT Filed: Apr. 10, 2024
(86) PCT No.: PCT/EP2024/059688

§ 371 Date: Oct. 9, 2025

(30) Foreign Application Priority Data
Apr. 13, 2023 (EP) 23167815.2

(51) Int. Cl. A61M 60/242; A61M 60/178; A61M 60/422; A61M 60/859; A61M 60/863

(52) U.S. Cl. CPC A61M 60/242; A61M 60/178; A61M 60/422; A61M 60/859; A61M 60/863

(57) ABSTRACT

The present disclosure relates to a blood pump (10) comprising a pumping section (12) having a blood flow inlet (14) and a blood flow outlet (16), an axial pump (18) having a longitudinal axis (LA) and at least partially being disposed within the pumping section (12). The axial pump (18) has a motor (20) and a pump element (22) and the motor (20) is configured to drive the pump element (22) so that a flow is generated between the blood flow inlet (14) and the blood flow outlet (16) of the pumping section (12). The pumping section (12) comprises a main portion (24) and an inlet portion (26). The inlet portion (26) is configured to be placed within a space of a patient's heart. The blood pump (10) further comprises a radially extending attachment portion (28) disposed about the main portion (24), and the attachment portion (28) is configured to be attached to an outer surface of the patient's heart. The present disclosure further relates to a blood pump system comprising a blood pump.

### BACKGROUND OF THE INVENTION

Various blood pumps are known from the prior art and are also called ventricular assist devices. A blood pump assists an impaired heart by conveying blood e.g., from the left ventricle to the aorta. Some blood pumps may be introduced percutaneously during a cardiac procedure through the vascular system, such as by a catheterization procedure e.g., the blood pumps of the Impella® family of the applicant. Those blood pumps are also called intracardiac or intravascular blood pumps, as they reach through a heart valve and form a direct connection, for example between the left ventricle and the aorta or the right ventricle or right atrium and the pulmonary artery. Of course, it is also possible to implement an unloading of blood from the venous system into the patient's heart.

Other blood pumps are introduced during a surgical procedure and bypass the heart valve in that the blood pump is substantially located outside of the heart. A part of the blood pump reaches through the heart wall and the blood pump is further connected to the respective blood vessel thereby bypassing the respective heart valve. Such blood pumps are also called extracardiac blood pump. The extracardiac blood pumps known from the prior art use centrifugal pumps for conveying the blood. However, those blood pumps are of comparably large size and therefore restricted in the medical application. In particular, a pediatric application is often impossible due to the reduced space available in the thorax of the pediatric patient. In other cases, a pediatric application of those blood pumps might not be wanted because the application of those pumps requires to punch out an opening in the heart which is particularly large in comparison to the small heart of the pediatric patient.

Hence, it is an objective of the present disclosure to provide a relatively small blood pump. It is further an objective of the present disclosure to provide further improvements to the know blood pumps. It is a further objective of the present disclosure to provide a blood pump system which allows for a facilitated placement of the blood pump within the patient.

### SUMMARY OF THE INVENTION

According to a first aspect, a blood pump is provided comprising a pumping section and an axial pump. The axial pump has a longitudinal axis. The pumping section may have a blood flow inlet and a blood flow outlet. The axial pump may at least partially be disposed within the pumping section. The axial pump may have a motor, in particular an electric motor, and a pump element, wherein the motor is configured to drive the pump element so that a flow is generated between the blood flow inlet and the blood flow outlet of the pumping section. The pumping section may comprise a main portion and an inlet portion, wherein the inlet portion is configured to be placed within a space of a patient's heart. The space may for instance be the left ventricle or the right ventricle, the left atrium or the right atrium. The blood pump may further comprise a radially extending attachment portion disposed about the main portion, wherein the attachment portion is configured to be attached to an outer surface of the patient's heart.

Thus, the blood pump according to the present disclosure may be an extracardiac blood pump having an axial pump. This configuration allows for a particular small pumping section having an outer diameter of 12 mm or less. In addition, the length of the pumping section may be 35 mm or less. Therefore, the blood pump according to the present invention can be more easily placed within a pediatric patient's thorax.

The pumping section may be a pump housing. The pump element may be an impeller. The inlet portion may be arranged concentrically with the longitudinal axis of the axial pump. Preferably, a longitudinal axis of the pumping section coincides with the longitudinal axis of the axial pump.

The blood flow inlet may be provided on the inlet portion and the blood flow outlet may be provided on the main portion.

Although a part of the blood pump according to the present invention is labeled as an inlet portion, a blood flow may also be generated in a flow direction towards the inlet portion e.g., when the blood pump is configured to unload blood from the patient's blood vessel system to the patient's heart.

Preferably, a connective pathway which connects the blood flow inlet and the blood flow outlet is formed in the pumping section. The connective pathway may partially be formed by an annular gap. The annular gap may be defined by an inner peripheral surface of the pumping section and at least a part of the axial pump, in particular between the inner peripheral surface of the pumping section and an outer peripheral surface of a housing of the axial pump.

The main portion may comprise a pump mounting portion extending radially inwardly from an inner peripheral surface of the main portion. The motor of the axial pump may comprise a magnetic circuit jacket disposed partially in the pump mounting portion. This allows for a particularly compact design and an optimal blood flow between the blood flow inlet and the blood flow outlet. The magnetic circuit jacket may at least partially form a housing of the axial pump. The magnetic circuit jacket may be made of a ferritic iron-chromium-aluminum alloy i.e., of a FeCrAl alloy. The ferritic iron-chromium-aluminum alloy may comprise between 1% and 7% of aluminum (Al). The ferritic iron-chromium-aluminum alloy may comprise between 20% and 25% of chromium (Cr). Preferably, the ferritic iron-chromium-aluminum alloy comprises 5.8% of Al and/or preferably between 20.5% and 23.5% of Cr. Further, the ferritic iron-chromium-aluminum alloy preferably comprises between 0% and 0.08% of carbon (C), between 0% and 0.7% of silicon (Si) and/or between 0% and 0.4% of Manganese (Mn). This allows for a superior form stability. Further, said material has superior oxidation properties and has a low tendency to ageing and a low resistance change. The ferritic iron-chromium-aluminum alloy may be Kanthal APM.

The magnetic circuit jacket may have a smaller diameter portion and a larger diameter portion. The larger diameter portion may be disposed in the pump mounting portion. This allows for an optimal blood flow along the motor.

The magnetic circuit jacket may comprise a tapered transition portion between the smaller diameter portion and the larger diameter portion. The tapered transition portion may in particular have a smooth gradient to allow for optimal blood flow.

The main portion may comprise an axially extending clamping portion on its outer peripheral surface. The attachment portion may be detachably mounted to the clamping portion, so that the attachment portion may be attachable at any position along the axial extension of the clamping portion. The clamping portion is preferably concentric with the longitudinal axis of the axial pump. Accordingly, the position of the blood pump can be individually adapted to the patient by mounting the attachment portion at different positions along the axial extension of the clamping portion. Thus, the axial distance from the attachment portion to the inlet portion can be adjusted. In other words, it is adjustable how far the inlet portion extends into the space of the patient's heart.

The attachment portion may comprise a slotted annular member and a tangential fastener. The tangential fastener may be tangential to a circle that is concentric with the longitudinal axis of the axial pump. The slotted annular member may be disposed about the main portion. The slotted annular member may comprise a first circumferential end and a second circumferential end being spaced from each other, so that a slot is formed between the first circumferential end and the second circumferential end. The tangential fastener may be configured to move the first circumferential end relative to the second circumferential end so that the size of the slot is variable. Narrowing the slot results in a clamping force clamping the attachment portion to the main portion, as an area spanned by the annular member being perpendicular to the longitudinal axis decreases. In particular, the tangential fastener may be a screw and tightening the tangential fastener narrows the slot. Conversely, loosening the tangential fastener reduces the clamping forces and allows to detach the attachment portion from the main portion.

A sealing element may be disposed in the slot. The sealing element is preferably an elastic sealing element. Preferably, the sealing element is made from a biocompatible material. The sealing element seals the slot and thus hinders bypass blood flow from the space of the patient's heart along the outer peripheral surface of the pumping section.

The tangential fastener preferably extends through the sealing element. In other words, the sealing element may be disposed about an outer surface of the tangential fastener. The position of the sealing element is thus securely set.

The pumping section may be a multipart member, wherein the main portion may comprise a first main portion part and second main portion part. The first main portion part may be made of a metallic material, preferably of titanium or a titanium alloy. The second main portion part may be made of a plastic material, preferably of polycarbonate. Of course, the first main portion part may also be made of a plastic material, preferably of polycarbonate, or of a different metallic material. Accordingly, the second main portion part may also be made of a metallic material, preferably of titanium or a titanium alloy. The first main portion part and the second main portion part may be attached to each other by suitable methods, like gluing, clamping, bonding or welding. The inlet portion may be formed separately from the main portion or may be formed integrally with the main portion as a one-piece unitary member. This allows for a facilitated assembly of the blood pump and can further reduce the costs by choosing a respective material combination.

Preferably, the blood flow outlet is provided on the second main portion part. The blood flow outlet may comprise a first spout. The first spout may extend tangentially to a circle that is concentric with the longitudinal axis of the axial pump. This allows for a particularly compact design of the blood pump.

The blood pump may further comprise a tubular graft connected to the first spout. The tubular graft may be configured to be attached to a patient's blood vessel e.g., to the aorta of the patient.

The blood pump may further comprise a fixation member and a bushing with a circumferential undercut. The bushing may be disposed about the first spout. The tubular graft may be disposed about the bushing. In particular, an axial end of the tubular graft may be disposed about the bushing. The fixation member may be configured to clamp the tubular graft to the circumferential undercut. This allows for an easy and secure mounting and demounting of the tubular graft to the pumping section.

An exoskeleton may be disposed about the tubular graft. The exoskeleton can hinder any damage to the tubular graft and can further allow better placement of the tubular graft within the thorax of the patient.

The exoskeleton may comprise a plurality of hingewise coupled first members. The first members may all be formed identically. Each first member may be composed of an annular base member and two attachment protrusions disposed on opposite sides of the annular base member and two attachment openings disposed on opposite sides of the annular base member. The attachment protrusions may be provided on respective lugs. The attachment openings may be provided on respective lugs. The attachment protrusions protrude radially outwardly from the annular base member. The attachment protrusions and the attachment openings are offset by 180°. In other words, seen in a circumferential direction of the annular base member there is provided an attachment protrusion or an attachment opening every 90°, whereby an attachment opening is always located between the attachment protrusions. Hence, the attachment protrusions of one first member may be connected to the attachment openings of another first member thereby forming a hinge.

Preferably, the first member being closest to the first spout is hingewise coupled to the pumping section. Preferably, this first member is hingewise coupled to the fixation member.

The exoskeleton may further comprise a second member hingewise coupled to the first member farthest from the first spout. The second member does not comprise the two attachment openings, but only the two attachment protrusions. The attachment protrusions may be provided on respective lugs. A sleeve may be coupled to the second member, wherein the sleeve is disposed about the tubular graft. The sleeve can prevent buckling of the tubular graft.

The second member may comprise an axial through hole and the tubular graft may extend through the axial through hole. The diameter of the axial through hole may at least partially reduce in a direction towards the first spout. In other words, the diameter of the axial through hole widens in a direction away from the first spout. Preferably, the diameter change is a smooth transition. Thus, no sharp corner or the like is formed which may damage the tubular graft or which may promote buckling of the tubular graft.

A first sensor may be disposed in proximity to the blood flow outlet, preferably within the pumping section. The first sensor may be a first optical sensor. The first sensor may be configured to sense a pressure parameter, in particular a pressure parameter being an indication for the pressure in the blood vessel the tubular graft is attached to. The blood vessel may be the aorta and the pressure parameter may thus deliver an indication for the aortic pressure.

A second sensor may be disposed in proximity to the blood flow inlet. The second sensor may be a second optical sensor. The second sensor may be configured to sense a pressure parameter, in particular a pressure parameter being an indication for the pressure in the space of the heart. The first pressure sensor may be attached to the inlet portion and may for instance sense a pressure parameter delivering an indication for the left ventricular pressure.

Preferably, the pressure parameters are transmitted to a control unit which is configured to modulate a rotational speed of the pump element. The control unit may be part of the blood pump or may be part of a blood pump system comprising the blood pump. Based on the pressure parameters transmitted, the control unit may calculate a bandwidth of motor currents applied to the motor. The motor currents essentially correspond to a rotational speed of the motor. In particular, the control unit may be configured to modulate the motor currents applied to the motor and hence, the rotational speed of the pump element over time so that an overall blood flow is achieved that matches with the expected blood flow of a heart. The control unit may be configured to modulate the motor currents so that a continuous total blood flow or a pulsed total blood flow is achieved. In particular, the rotational speed of the pump element may be in a range between 17,000 rpm and 33,000 rpm, preferably in a range between 20,000 rpm and 26,000 rpm. Accordingly, the motor currents may be in a range between 100 mA and 700 mA, preferably in a range between 150 mA and 380 mA.

A diameter of the main portion of the pumping section may be larger than a diameter of the inlet portion of the pumping section. The pumping section may comprise a tapered portion connecting the main portion and the inlet portion of the pumping section. This greatly facilitates placement of the blood pump and in particular facilitates placement of the inlet portion within the space of the patient's heart.

The blood flow inlet of the pumping section may comprise at least one radial inlet opening, preferably a plurality of radial inlet openings. In particular, the blood flow inlet may comprise five radial inlet openings. The plurality of radial inlet openings are preferably evenly distributed around the circumference of the inlet portion. The blood flow inlet preferably further comprises an axial inlet opening. This warrants that no suction of tissue over time of use of the blood pump can occur, which might otherwise impair the functionality of the blood pump.

The axial pump may comprise a drive shaft driven by the motor, wherein the pump element is supported on the drive shaft. A rotor of the motor may be supported on the drive shaft, so that the rotor is disposed radially inwardly of a stator of the motor. Alternatively, the pump element may be supported on a drive assembly, wherein the drive assembly comprises magnets which form a rotor of the motor and which are disposed adjacent to electrically magnetized zones in a stator of a drive section of a motor. Alternatively, the pump element may comprise the magnets which are disposed adjacent to the electrically magnetized zones in the stator of the drive section. Based on the attractive force between the magnets of the drive assembly or the pump element and the magnetized zones in the stator of the drive section, rotation can be transmitted to the drive assembly and hence, to the pump element or directly to the pump element. In particular, a rotating magnetic field can be established within the stator of the drive section which rotates the drive assembly or the pump element in that the control unit applies appropriate current or voltage respectively to the stator in a controlled manner. Hence, the pump element can be driven in a contactless fashion.

In case the pump element is driven contact free, the axial pump may comprise a bearing assembly supporting the pump element or the drive assembly, respectively. The bearing assembly may comprise a first bearing disposed in the inlet portion at a first axial end of the pump element. The bearing assembly may comprise a second bearing disposed at a second axial end of the pump element. An according bearing assembly is disclosed in PCT/EP2023/051785 (application number) which is hereby incorporated by reference in its entirety for all purposes.

The pumping section may comprise a second spout extending tangentially to a circle that is concentric with the longitudinal axis of the axial pump. The second spout may be configured to guide and/or support an elongated connection assembly. Alternatively, the pumping section may comprise a guide opening extending tangentially to a circle that is concentric with the longitudinal axis of the axial pump through the pumping section, in particular through the main portion and preferably through the second main portion part. The guide opening may be configured to guide and/or support the elongated connection assembly. The elongated connection assembly may comprise one or more of a catheter, a motor cable, a first optical fiber and a second optical fiber. The second spout or the guide opening respectively may be arranged in parallel to the first spout. The second spout or the guide opening may have an orientation identical to the first spout.

The main portion may comprise a circumferential section with an increased surface roughness. The circumferential section with the increased surface roughness is preferably generated by material ablation, preferably by laser material ablation, or preferably by laser bead blasting. Preferably, the circumferential section is surrounded by patient's tissue when the blood pump is placed within the patient. In particular, the circumferential section may be surrounded by a portion of the patient's heart when the blood pump is placed within the patient.

Preferably, the main portion of the pumping section comprises a central mounting opening at an axial end opposite to the inlet portion. Preferably, a cover closes the central mounting opening. The cover may be made of a plastic material, in particular of polycarbonate.

Preferably, the central mounting opening comprises an annular surface recessed in a direction towards the inlet portion. The annular surface may be flush with an axial end face of the axial pump, in particular with an axial end face of the magnetic circuit jacket. This allows to define the correct mounting position of the axial pump.

According to a second aspect, a blood pump assembly comprises a blood pump as described above and an expandable member. The expandable member may be configured to be disposed within the inlet portion at a free axial end of the inlet portion. The expandable member may be an inflatable and deflatable balloon. During placement of the blood pump, the inflated expandable member is disposed in the inlet portion of the blood pump so that it is the farthest outward part. When penetrating the patient's punctured heart, the expandable member widens the puncture and facilitates the placement of the inlet portion within the patient's heart. After placement of the blood pump, the expandable member is deflated and may be removed from the patient's body e.g., by using a guidewire or a hose configured for inflation and deflation of the expandable member. Preferably, the expandable member is inflated with a liquid, preferably with a NaCl-solution.

The expandable member preferably comprises a base portion and a tip portion. The tip portion may have a conical shape. The base portion may be provided in the inlet portion when the expandable member is inflated. The tip portion may extend from the axial inlet opening when the expandable member is inflated. The expandable member may comprise a central opening for a guide wire to pass through.

Thus, a method for placing the blood pump assembly according as described above may comprise the following step of puncturing the patient's heart; and placing at least a portion of the blood pump assembly through the puncture. The step of placing at least a portion of the blood pump assembly through the puncture may comprise introducing a guide wire through the puncture. The step of placing at least a portion of the blood pump assembly through the puncture may comprises dilatating the puncture. The step of placing at least a portion of the blood pump assembly through the puncture may comprise inflating the expandable member, preferably with a liquid e.g., with a NaCl-solution. The step of placing at least a portion of the blood pump assembly through the puncture may comprise advancing the blood pump along the guide wire. The step of placing at least a portion of the blood pump assembly through the puncture may comprise moving the tip portion of the expandable member through the puncture. The step of placing at least a portion of the blood pump assembly through the puncture may comprise deflating and removing the expandable member after the inlet portion is received within the space of the patient's heart.

Thus, according to the inventive method, there is no need to remove tissue for placement of the blood pump. Rather, the patient's heart is punctured with the puncture being dilatated. Accordingly, recovery of the patient's heart is greatly facilitated after removal of the blood pump, in particular when the patient is a pediatric patient.

### BRIEF DESCRIPTION OF THE DRAWINGS

The foregoing summary as well as the following detailed description of preferred embodiments will be better understood when read in conjunction with the appended drawings. For the purpose of illustrating the present disclosure, reference is made to the drawings. However, the scope of the disclosure is not limited to the specific embodiments disclosed in the drawings.

In the Drawings:

FIG. 1 is a first perspective view of a blood pump according to a first embodiment,

FIG. 2 is a second perspective view of the blood pump of FIG. 1,

FIG. 3 is a sideview of the blood pump of FIG. 1,

FIG. 4 is a top view of the blood pump of FIG. 1,

FIG. 5 is a back view of the blood pump of FIG. 1 with removed cover,

FIG. 6 is a cross section of the blood pump along the line A-A shown in FIG. 4,

FIG. 7 is a modified view of the blood pump of FIG. 6,

FIG. 8 is a perspective view of a pumping section of the blood pump of FIG. 1,

FIG. 9 is a side view of the pumping section of FIG. 8,

FIG. 10 is a perspective view of an attachment portion of the blood pump of FIG. 1,

FIG. 11 is a front view of the attachment portion of FIG. 10,

FIG. 12 is a side view of a blood pump according to a second embodiment,

FIG. 13 is a front view of the blood pump of FIG. 12,

FIG. 14 is a partial cross section of the blood pump of FIG. 12,

FIG. 15 is a cross section of the blood pump of FIG. 14,

FIG. 16 is a perspective view of a blood pump assembly comprising a blood pump of FIG. 1, and

FIG. 17 is a cross section of the blood pump assembly of FIG. 16.

### DETAILED DESCRIPTION

In the Figures, like reference numbers denote similar or identical elements. Further, for lucidity reasons not all reference numbers are depicted in every Figure.

### First Exemplary Embodiment

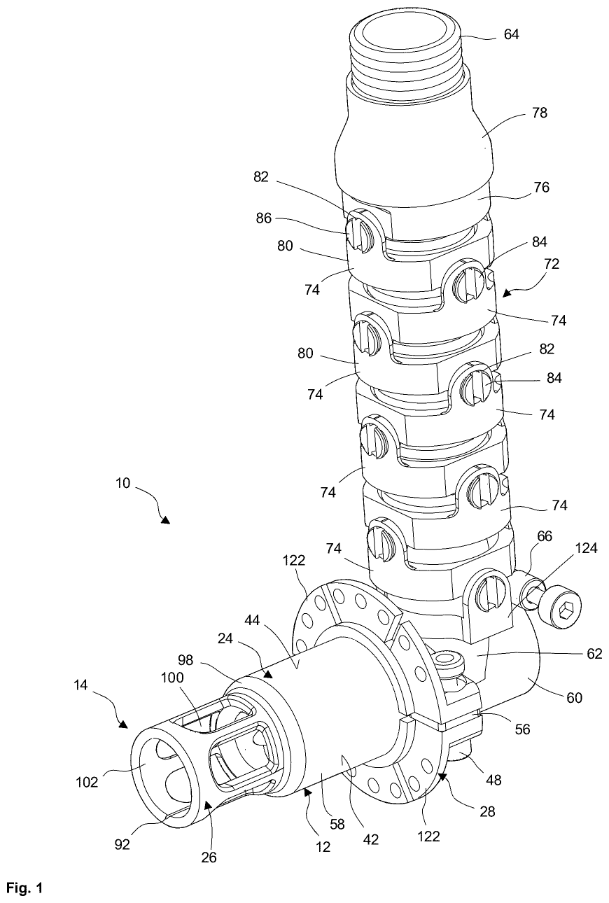

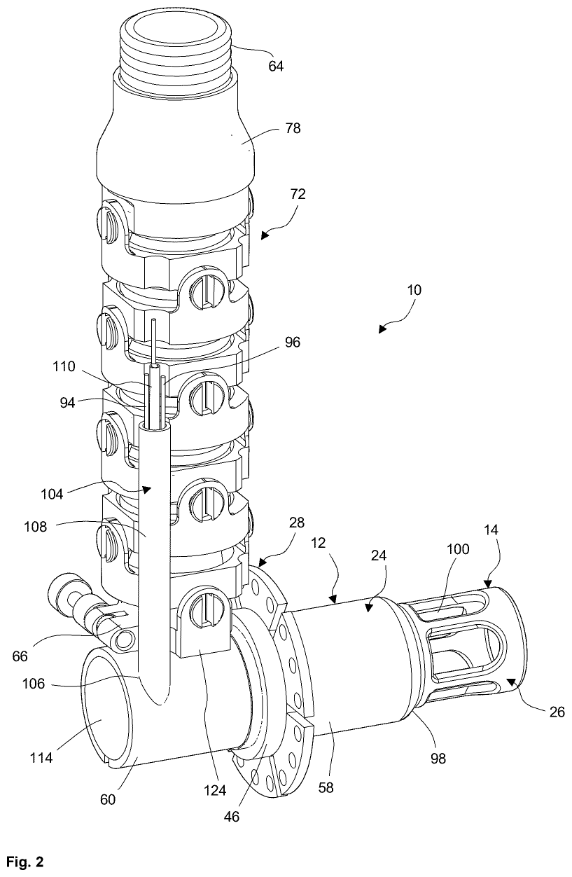

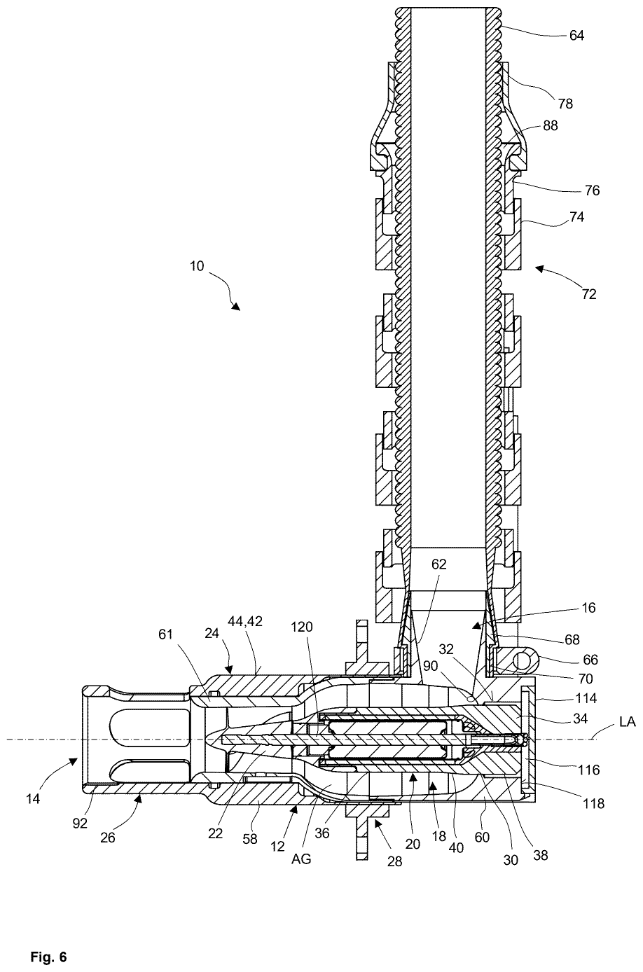

FIGS. 1 and 2 show perspective views of a blood pump 10 according to a first exemplary embodiment. The blood pump 10 comprises a housing in form of a pumping section 12. The pumping section 12 is essentially cylindrical and has a blood flow inlet 14 and a blood flow outlet 16 (see e.g., FIG. 6). The pumping section 12 comprises a main portion 24 and an inlet portion 26. The blood flow inlet 14 is disposed on the inlet portion 26. The main portion 24 comprises the blood flow outlet 16. The main portion 24 and the inlet portion 26 are connected by a tapered portion 98. The inlet portion 26 is configured to be placed within the space of a patient's heart, when the blood pump 10 is placed within a patient. For instance, in case the blood pump 10 is configured as a left ventricular support device, the space of the patient's heart is the left ventricle. Generally, the heart is then punctuated in the area of the apex of the heart and the inlet portion 26 is gently pushed through the punctation to extend through the patient's heart into the left ventricle, as will be described in more detail below.

The blood flow inlet 14 comprises an axial inlet opening 102 and a plurality of radial inlet openings 100 evenly distributed along the circumference of the inlet portion 26. In the first exemplary embodiment, the blood flow inlet 14 comprises five radial inlet openings 100.

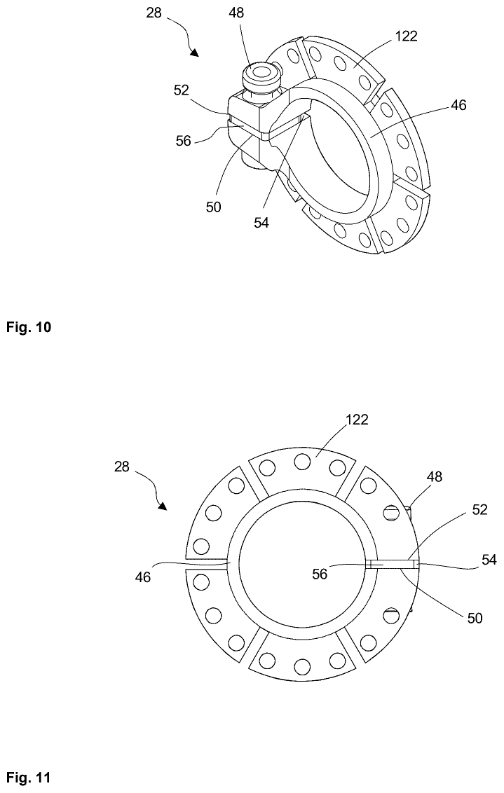

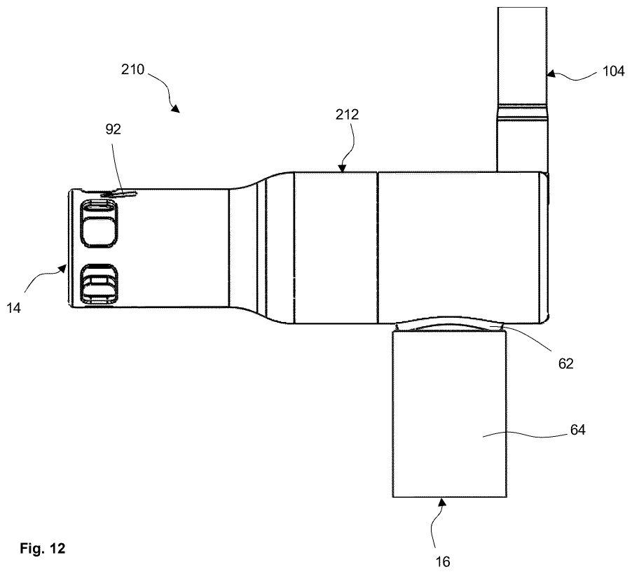

Furthermore, the blood pump 10 comprises a radially extending attachment portion 28 which is disposed about the main portion 24. The attachment portion 28 is configured to be attached to an outer surface of the patient's heart. In particular, the attachment portion 28 may be attached to a felt, a fabric, a textile, a tissue, a non-woven or the like (not shown, in the following referred to as felt) which is attached to the outer surface of the patient's heart. In particular, the attachment portion 28 is sewn to the felt or directly to the outer surface of the patient's heart. The attachment portion 28 will be described in more detail below with particular reference to FIGS. 10 and 11.

### Axial Pump 18

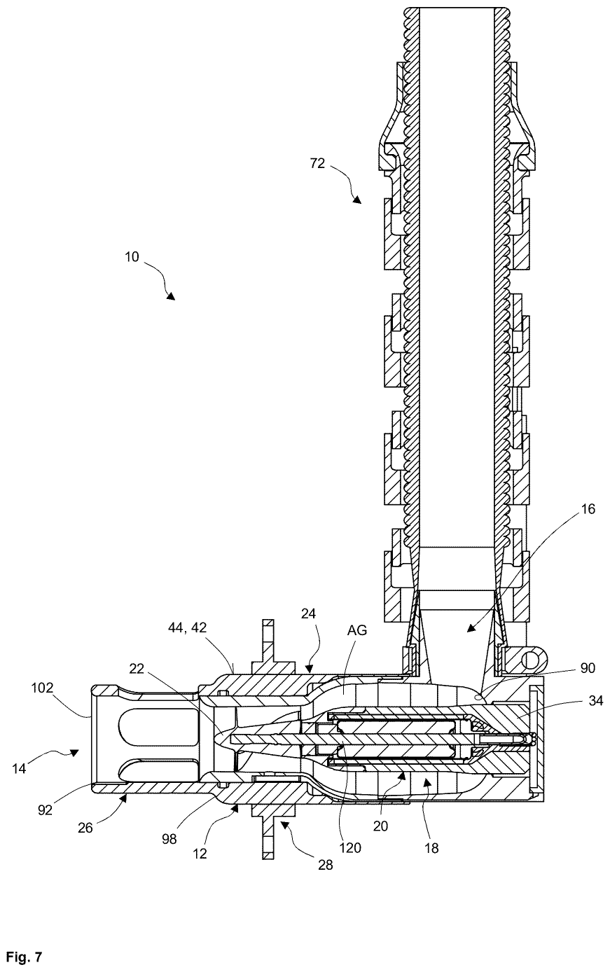

As depicted in FIGS. 6 and 7, the blood pump 10 further comprises an axial pump 18. The axial pump 18 has a motor 20 and a pump element 22 in form of an impeller. The axial pump 18 defines a longitudinal axis LA which coincides with a longitudinal axis of the pumping section 12. The motor 20 is configured to drive the pump element 22 so that a blood flow is generated between the blood flow inlet 14 and the blood flow outlet 16. The motor 20 is configured essentially similar to the motor disclosed in WO 2008/116765 A2 which is hereby incorporated by reference in its entirety for all purposes. The motor 20 comprises a magnetic circuit jacket 34. In the embodiment of FIGS. 6 and 7, the magnetic circuit jacket 34 is not completely circular cylindrical along its axial extension, but comprises a smaller diameter portion 36 and a larger diameter portion 38 which are connected by a tapered transition portion 40. The tapered transition portion 40 allows for a smooth transition between the smaller diameter portion 36 and the larger diameter portion 38. The smaller diameter portion 36 is orientated in direction to the inlet portion 26 and the larger diameter portion 40 in a direction away from the inlet portion 26.

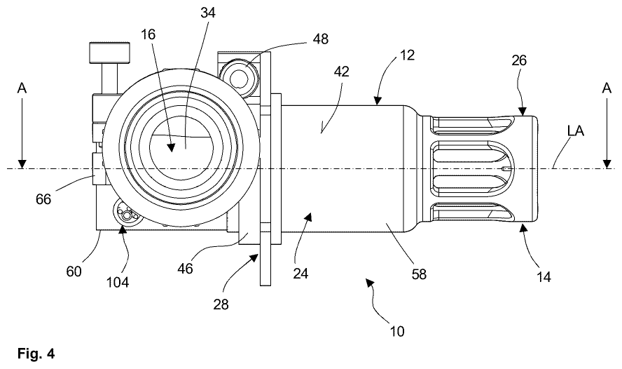

When the pump element 22 rotates at a given rotational speed, blood is sucked into the pumping section 12 via the blood flow inlet 14 i.e., via the radial inlet openings 100 and the axial inlet opening 102. The so generated blood flow is first of all essentially parallel to the longitudinal axis LA and flows from the blood flow inlet 14 through an annular gap AG defined by an inner peripheral surface 32 of the main portion 24 and the axial pump 18. Then, the blood flow is deflected towards the blood flow outlet 16, for example in a tangential direction. The annular gap AG is part of a connective pathway connecting the blood flow inlet 14 and the blood flow outlet 16, as can be seen in FIG. 6, for example. As depicted in FIG. 4, the blood flow outlet 16 is tangentially orientated with respect to the longitudinal axis LA.

### Main Portion 24 And Inlet Portion 26

The main portion 24 is a multipart member and comprises a first main portion part 58, a second main portion part 60 and a third main portion part 61. The first main portion part 58 is integrally formed with the inlet portion 26 and the tapered portion 98. The first main portion part 58 is made of a metallic material, and in particular of titanium or a titanium alloy. The second main portion part 60 is attached to an axial end of the first main portion part 58 opposite the inlet portion 26. The second main portion part 60 is made of a plastic material, and in particular of polycarbonate. The first main portion part 58 and the second main portion part 60 are fixed to each other via gluing. The third main portion part 61 is disposed radially inwardly within the first main portion part 58. The third main portion part 61 is designed in such a way that the blood flow is as unobstructed as possible. As shown in FIGS. 6 and 7 the third main portion part 61 covers edgy areas on the inner peripheral surface of the first main portion part 58. Third main portion part 61 is made of a plastic material, in particular of polycarbonate. The third main portion part 61 is fixed to the first main portion part 58 and the third second portion part 60 via gluing.

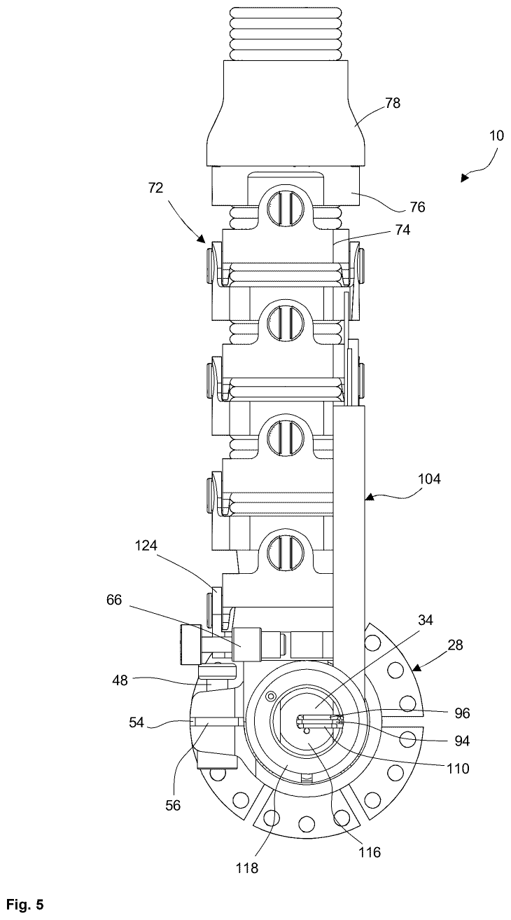

The axial pump 18 is mounted to a pump mounting portion 30 extending radially inwardly from the inner peripheral surface 32 of the main portion 24. In particular, the pump mounting portion 30 extends radially inwardly from the inner peripheral surface of the second main portion part 60 and the larger diameter portion 38 of the magnetic circuit jacket 34 of the motor 20 is supported within the pump mounting portion 30. To mount the axial pump 18, the main portion 24 comprises a central mounting opening 116 at a free axial end opposite to the inlet portion 26. The central mounting opening 116 comprises an annular surface 118 recessed in a direction towards the inlet portion 26. When mounting the axial pump 18, it is pushed into the main portion 24 through the central mounting opening 116 until the annular surface 118 is flush with the respective axial end face of the axial pump 18 or magnetic circuit jacket 34 respectively. The axial pump 18 is further attached to the pump mounting portion 30 via gluing. Thereafter, a cover 114 is attached to close the central mounting opening 116 via gluing. In the back view shown in FIG. 5, the cover 114 is not displayed.

In addition, the main portion 24 comprises a circumferential section 112 with an increased surface roughness. The circumferential section 112 with the increased surface roughness is preferably generated by material ablation, preferably by laser material ablation, or preferably by laser bead blasting. The circumferential section 112 is surrounded by the patient's heart tissue when the blood pump 10 is placed within the patient, which enhances the attachment between the blood pump 10 and the patient's heart.

### Attachment Portion 28

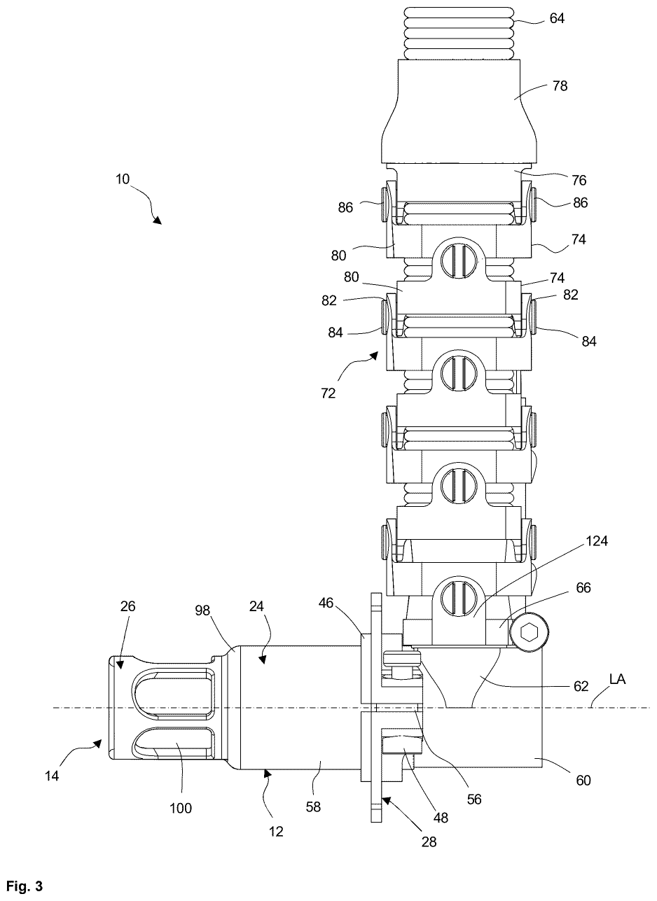

As shown in FIGS. 1 to 7, 10 and 11, the attachment portion 28 comprises a slotted annular member 46, a tangential fastener 48 and a plurality of attachment flaps 122 extending radially outwardly from the annular member 46. The annular member 46 comprises a first circumferential end 50 and a second circumferential end 52 which face each other in the circumferential direction. A slot or gap 54 is formed between the first circumferential end 50 and second circumferential end 52. The first circumferential end 50 and the second circumferential end 52 can be moved relative to each other by tightening or releasing the tangential fastener 48. Thus, the circumferential extension of the slot 54 is variable.

Tightening the tangential fastener 48 reduces the size of an area spanned by the slotted annular member 46 being perpendicular to the longitudinal axis LA. Accordingly, the attachment portion 28 can be disposed about the main portion 24 fixed to an outer peripheral surface 44 of the main portion 24 by a clamping force induced by tightening the tangential fastener 48. Releasing the tangential fastener 48 largens the size of the area spanned by the slotted annular member 46 and detaches the annular member 46 from the outer peripheral surface 44 of the main portion 24. As a part of the outer peripheral surface 44 of the main portion 24 defines a clamping portion 42, the attachment portion 28 can be attached at any position along the axial extension of the clamping portion 42 by tightening and releasing the tangential fastener 28. This allows to adjust the axial extension of the portion extending into the space of the patient's heart. FIGS. 6 and 7 exemplarily depict two different positions of the attachment portion 28 along the clamping portion 42 of the main portion 24.

The attachment flaps 122 each comprise a plurality of axially extending through holes configured for attachment to the felt or the outer surface of the patient's heart in that a surgical suture may be passed through the holes. Furthermore, the attachment flaps 122 each have a space in between which reaches down to the slotted annular member 46. This is necessary to allow the first circumferential end 50 and the second circumferential end 52 of the slotted annular member 46 to be moved relative to each other via the tangential fastener 48.

Furthermore, the attachment portion 28 comprises a sealing element 56 sealing the slot 54. The sealing element 56 is a flexible sealing element composed of a biocompatible material. In this exemplary embodiment, the sealing element 56 is disposed within the slot 54 with the tangential fastener 48 extending through the sealing element 56. The sealing element 56 hinders bypass blood flow along the outer peripheral surface of the pumping section 12 when the blood pump 10 is placed within the patient.

### Blood Flow Outlet 16, Tubular Graft 64 And Exoskeleton 72

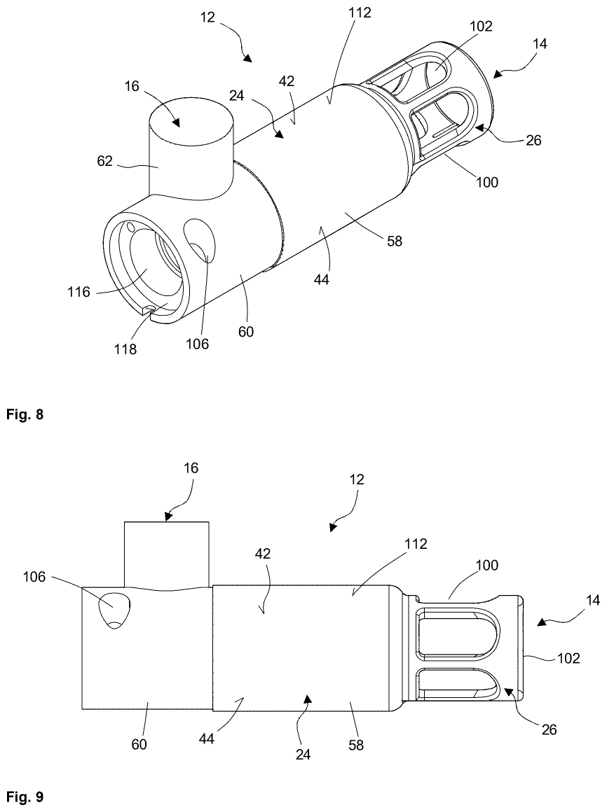

As depicted in FIGS. 8 and 9, the blood flow outlet 16 comprises a first spout 62 extending tangentially from the main portion 24 relative to the longitudinal axis LA. In this exemplary embodiment, the first spout 62 is provided on the second main portion part 60. The first spout 62 is configured to support a tubular graft 64 through which the blood pumped by the axial pump 18 is delivered to the patient's blood vessel. As mentioned above, this exemplary embodiment is configured as a left ventricular assist device, so that the tubular graft 64 is attached to the patient's aorta.

For fixation of the tubular graft 64 the blood pump 10 comprises a fixation member 66 and a bushing 68, cf. FIGS. 6 and 7. The bushing 68 is provided with a circumferential undercut 70. The bushing 68 is disposed about an outer peripheral surface of the first spout 62 so that the circumferential undercut 70 is orientated towards the origin of the first spout 62 on the main portion 24. The bushing 68 also comprises a tapered portion which facilitates putting the axial end of the tubular graft 64 over the bushing 68. The fixation member 66 is configured as a clamp and clamps the tubular graft 64 to the circumferential undercut 70. In this exemplary embodiment, the clamping force is generated by a fastener. Of course, the fixation member 66 can be configured differently as long as a sufficiently high attachment force can be generated.

To provide a sufficient stability of the tubular graft 64 when placed within the patient's body, the blood pump 10 comprises an exoskeleton 72 which extends from the fixation member 66 along a portion of the tubular graft 64. The exoskeleton 72 comprises a plurality of first members 74.

The first members 74 are identical and each comprises an annular base member 80, two attachment openings 82 and two attachment protrusions 84. The two attachment openings 82 are provided on two lugs extending axially from the annular base member 80 and being exactly opposite to each other. The two attachment protrusions 84 are also provided on two lugs also extending axially from the annular base member 80 in an opposite direction compared to the two lugs provided with the attachment openings 82 and also being exactly opposite to each other. Thus, the lugs are each offset by 90° along the circumference of the annual base member 80. As can be seen from e.g., FIG. 1 the plurality of first members 74 are hingewise attached to each other in that the attachment protrusions 84 of one first member 74 are supported in the attachment openings 82 of an adjacent first member 74. To connect the first members 74 to one another, the first members are always offset by 180°. In this exemplary embodiment, the exoskeleton 72 comprises a total of seven first members 74, but the exoskeleton 72 can also comprise more or fewer first members 74 depending on the conditions needed.

In addition, the exoskeleton 72 comprises one second member 76 attached to the first member 74 being farthest from the first spout 62. The second member 76 also comprises annular base member and two attachment protrusions 86 being provided on axially extending lugs being exactly opposite to each other. Hence, the second member 76 can be hingewise connected to the adjacent first member 74. A sleeve 78 is coupled to the second member 76 and the tubular graft 64 extends through the sleeve 78. Furthermore, the annular member of the second member 76 defines an axial through hole 88 disposed about the tubular graft 64. The diameter of the axial through hole 88 partially reduces smoothly in a direction towards the first spout 62, as shown in FIGS. 6 and 7. The sleeve 78 and the smoothly reducing diameter inhibit buckling or bending of the tubular graft 64.

To attach the exoskeleton 72 to the pumping section 12, the fixation member 66 comprises two axially extending lugs 124 each having an attachment opening configured to receive the respective attachment protrusion 84 of the first member 74 being closest to the pumping section 12. Therefore, the lugs 124 are provided exactly opposite on the fixation member 66.

### First Sensor 90 And Second Sensor 92

The blood pump 10 comprises a first sensor 90. The first sensor 90 is an optical sensor configured to sense pressure parameters. In particular, the first sensor 90 is intended to deliver pressure parameters giving an indication for the pressure prevailing in the blood vessel the tubular graft 64 is attached to. In this exemplary embodiment, with the blood pump 10 being configured as a left ventricular assist device, the first sensor 90 is thus configured to deliver pressure parameters giving an indication for the aortic pressure. Therefore, the first sensor 90 is disposed within the main portion 24 in close proximity to the blood flow outlet 16, see FIGS. 6 and 7. In this embodiment, the first sensor 90 is disposed adjacent to the pump mounting portion 30.

The blood pump 10 comprises a second sensor 92. The second sensor 92 is an optical sensor configured to sense pressure parameters. In particular, the second sensor 92 is intended to deliver pressure parameters giving an indication for the pressure prevailing in the space of the patient's heart, into which the inlet portion 26 extends. In this exemplary embodiment, with the blood pump 10 being configured as a left ventricular assist device, the second sensor 92 is thus configured to deliver pressure parameters giving an indication for the left ventricular pressure. Therefore, the second sensor 92 is disposed at the inlet portion 26 in close proximity to the blood flow inlet 14, see FIG. 1. In this embodiment, the second sensor 92 is disposed adjacent to the axial inlet opening 102.

The first sensor 90 is connected to a control unit (not shown) via a first optical fiber 94. The second sensor 92 is connected to the control unit via second optical fiber 96. The control unit is configured to compute the signals delivered by first sensor 90 and the second sensor 92 and to modulate a rotational speed of the axial pump 18 based thereon. Based on the pressure parameters transmitted, the control unit may calculate a bandwidth of motor currents applied to the motor 20. The motor currents essentially correspond to a rotational speed of the motor 20. In particular, the control unit is configured to modulate the motor currents applied to the motor 20 and hence, the rotational speed of the pump element 22 over time so that an overall blood flow is achieved that matches with the expected blood flow of a heart. The control unit is configured to modulate the motor currents so that a continuous total blood flow or a pulsed total blood flow is achieved. In particular, the rotational speed of the pump element 22 is in a range between 17,000 rpm and 33,000 rpm, preferably in a range between 20,000 rpm and 26,000 rpm. Accordingly, the motor currents are in a range between 100 mA and 700 mA, preferably in a range between 150 mA and 380 mA.

### Guide Opening 106

As shown in FIGS. 8 and 9 the pumping section 12 comprises a guide opening 106 extending tangentially to the longitudinal axis LA through the pumping section 12. In particular, the guide opening 106 extends through the main portion 24, namely through the second main portion part 60. The guide opening 106 is configured to guide and support an elongated connection assembly 104. In this exemplary embodiment, the elongated connection assembly 104 comprises a catheter 108, a motor cable 110 configured to apply the respective currents to the motor 20, the first optical fiber 94 and the second optical fiber 96. The guide opening 106 is arranged in parallel to the first spout 62. In this exemplary embodiment, the guide opening 106 has an orientation identical to the first spout 62 in particular, the catheter 108 is supported in the guide opening 106 and the motor cable 110, the first optical fiber 94 and second optical fiber 96 are disposed within the catheter 108. Furthermore, a purge fluid channel may also be provided e.g., within the catheter 108 to apply purge fluid to the interior of the pumping section 12 in a known manner.

### Second Embodiment

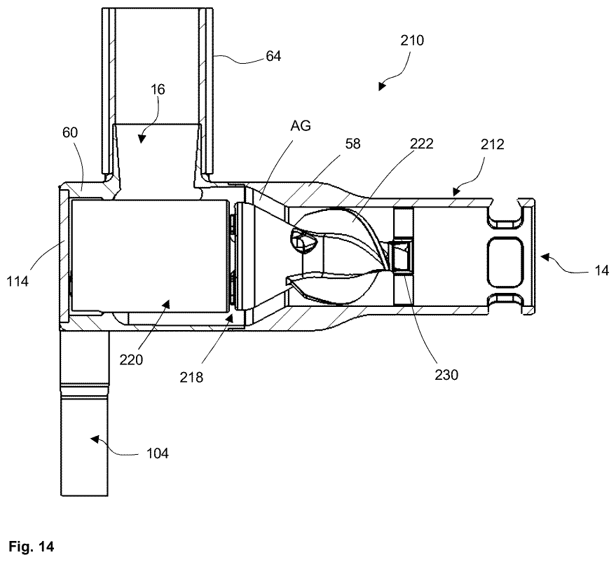

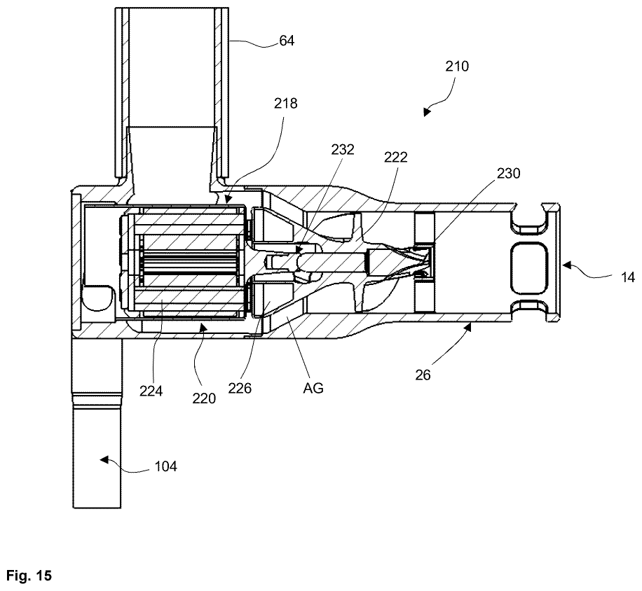

FIGS. 12 to 15 depict a second exemplary embodiment of a blood pump 210. The blood pump 210 differs from the blood pump 10 according to the first exemplary embodiment in that the axial pump 218 is configured differently. Furthermore, the second sensor 92 is provided on an outer circumferential surface of the blood flow inlet 14, as shown in FIG. 12. In addition, the pumping section 212 does not comprise a guide opening, but a second spout 228 extending tangentially from the pumping section 212.

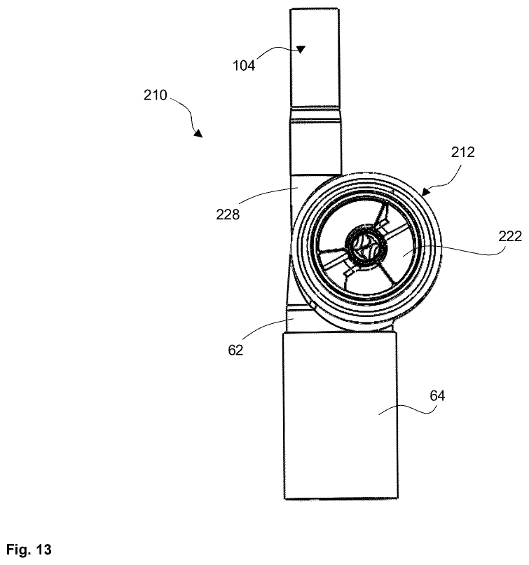

As depicted in FIG. 13, the second spout 228 extends in parallel to the first spout 62 but in an opposite direction. The second spout 228 is configured to guide and support the elongated connection assembly 104.

The axial pump 218 comprises a pump element 222 in form of an impeller which is driven contact free. Therefore, the pump element 222 comprises magnets 226 which form a rotor of the motor 220 and which are disposed adjacent to the electrically magnetized zones of a stator of a drive section 224 of the motor 220. The stator of the drive section 224 of the motor 220 is configured to generate a rotating magnetic field. The drive section 224 is disposed in the pump mounting portion 30 of the main portion 24 and configured to drive the pump element 222 in a contactless fashion. Based on the attractive force between the magnets 226 of the pump element 222 and the magnetized zones in the stator of the drive section 224, rotation can be transmitted directly to the pump element 222. In particular, the rotating magnetic field is established within the stator of the drive section 224 which rotates the pump element 222 in that the control unit applies appropriate current or voltage respectively to the stator of the drive section 224 in a controlled manner.

The axial pump 218 comprises a bearing assembly 230, 232 supporting the pump element 222. The bearing assembly comprises a first bearing 230 disposed in the inlet portion 14 at a first axial end of the pump element 222. The bearing assembly comprise a second bearing 232 disposed at a second axial end of the pump element 222. An according bearing assembly is disclosed in PCT/EP2023/051785 (application number) which is hereby incorporated by reference in its entirety.

### Blood Pump Assembly 300

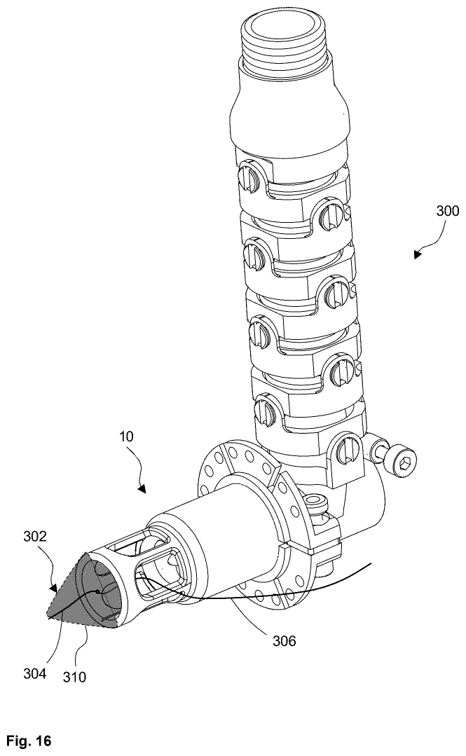

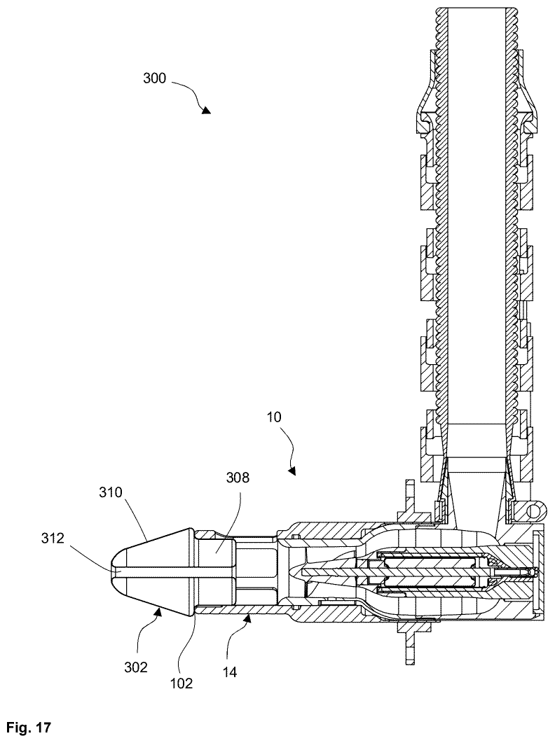

A blood pump assembly 300 is shown in FIGS. 16 and 17. The blood pump assembly 300 comprises a blood pump 10 according to the first embodiment or a blood pump 210 according to the second embodiment. Here, a blood pump 10 according to the first embodiment is exemplarily shown.

The blood pump assembly 300 may comprise the control unit, which may be provided extracorporeal. Furthermore, the blood pump assembly 300 comprises an expandable member 302 which is denoted by the dashed lines in FIG. 16. The expandable member 302 is an inflatable and deflatable balloon-like member which is disposed in the inlet portion 14 prior to the placement of the blood pump 10 within the patient's heart. In particular, the expandable member 302 is disposed in the axial inlet opening 102 to fully seal the axial inlet opening 102 when inflated and to be the most outward part.

For inflation and deflation of the expandable member 302 an inflation and deflation hose 306 is provided, which may be guided by a guidewire 304, as shown in FIG. 16.

FIG. 17 shows the expandable member 302 in the inflated state. The expandable member 302 comprises a base portion 308 and a tip portion 310. The base portion 308 is disposed within the inlet portion 14 and the tip portion 310 extends from the inlet portion 14 through the axial inlet opening 102. The tip portion has a conic shape. Further, the expandable member 302 comprises a central opening 312 to allow the guide wire (not shown in FIG. 17) to reach through the expandable member 302.

For placement of the blood pump 10, the patient's heart is generally punctured at the apex of the heart. Next the guide wire 304 is pushed through the punctation and a dilatator is guided along the guide wire until it is disposed within the punctation. Next, the punctation is widened by the dilatator to e.g., 26 French. The dilatator is then removed again and the blood pump 10 with the inflated expandable member 302 is advanced along the guide wire. The inlet portion 14 of the blood pump 10 is introduced into the space of the patient's heart through the punctation. To avoid any injury during insertion and to facilitate placement of the blood pump 10, the flexible expandable member 302 is provided. In particular, the conical shape of the tip portion 310 of the expandable member 302 allows for an eased insertion through the punctation, as the pre-dilatated puncture is again widened by the expandable member 302.

After placement of the blood pump 10 within the patient, the expandable member 302 is deflated and gently removed by pulling it out via the guidewire 304 or the inflation and deflation hose 306. As the expandable member 302 only has small dimensions in the deflated state, it can be pulled out from the patient's heart via the punctation without any problems.

### Exemplary Implementations

As already described, the technology described herein may be implemented in various ways. In that regard, the foregoing disclosure is intended to include, but not be limited to, the systems, methods, and combinations and sub-combinations thereof that are set forth in the following exemplary implementations. Preferred embodiments are described in the following paragraphs:
A1 Blood pump comprising: a pumping section having a blood flow inlet and a blood flow outlet, a pump at least partially being disposed within the pumping section, the pump having a motor and a pump element, the motor being configured to drive the pump element so that a flow is generated between the blood flow inlet and the blood flow outlet of the pumping section, wherein the pumping section comprises a main portion and an inlet portion, and wherein the inlet portion is configured to be placed within a space of a patient's heart, wherein the blood pump further comprises an attachment portion disposed about the main portion, and wherein the attachment portion is configured to be attached to an outer surface of the patient's heart.
A2 Blood pump according to paragraph A1, wherein the pump is an axial pump having a longitudinal axis.
A3 Blood pump according to paragraph A1 or A2, wherein the pumping section is a pump housing.
A4 Blood pump according to any one of the preceding paragraphs A1 to A3, wherein the pump element is an impeller.
A5 Blood pump according to any one of the preceding paragraphs A1 to A4, wherein the pumping section has a longitudinal axis, wherein the longitudinal axis of the pumping section preferably coincides with the longitudinal axis of the axial pump.
A6 Blood pump according to any one of the preceding paragraphs A1 to A5, wherein the blood flow inlet is provided on the inlet portion and/or wherein the blood flow outlet is provided on the outlet portion.
A7 Blood pump according to any one of the preceding paragraphs A1 to A6, wherein the main portion comprises a pump mounting portion extending radially inwardly from an inner peripheral surface of the main portion.
A8 Blood pump according to paragraph A7, wherein the pump is disposed in the pump mounting portion.
A9 Blood pump according to any one of the preceding paragraphs A1 to A8, wherein the motor comprises a magnetic circuit jacket.
A10 Blood pump according to paragraph A7 or A8, wherein the motor comprises a magnetic circuit jacket disposed in the pump mounting portion or a drive section disposed in the pump mounting portion and configured to drive the pump element in a contactless fashion.
A11 Blood pump according to paragraph A9 or A10, wherein the magnetic circuit jacket comprises a smaller diameter portion and a larger diameter portion.
A12 Blood pump according to paragraph A11, wherein the magnetic circuit jacket comprises a tapered transition portion between the smaller diameter portion and the larger diameter portion.
A13 Blood pump according to paragraph A11 or A12, wherein the larger diameter portion is disposed in the pump mounting portion.
A14 Blood pump according to any one of the preceding paragraphs A1 to A13, wherein the attachment portion is a radially extending attachment portion.
A15 Blood pump according to any one of the preceding paragraphs A1 to A14, wherein the main portion comprises an axially extending clamping portion on the outer peripheral surface.
A16 Blood pump according to paragraph A15, wherein the attachment portion is detachably mounted to the clamping portion, so that the attachment portion is attachable at any position along the axial extension of the clamping portion.
A17 Blood pump according to any one of the preceding paragraphs A1 to A16, wherein the attachment portion comprises a slotted annular member and a tangential fastener.
A18 Blood pump according to paragraph A17, wherein the slotted annular member is disposed about the main portion and comprises a first circumferential end and a second circumferential end being spaced from each other, so that a slot is formed between the first circumferential end and the second circumferential end.
A19 Blood pump according to paragraph A18, wherein the tangential fastener is configured to move the first circumferential end relative to the second circumferential end so that the size of the slot is variable.
A20 Blood pump according to paragraph A18 or A19, wherein a sealing element is disposed in the slot.
A21 Blood pump according to paragraph A20, wherein the tangential fastener extends through the sealing element.
A22 Blood pump according to paragraph A20 or A21, wherein the sealing element is made of an elastic material.
A23 Blood pump according to any one of the preceding paragraphs A1 to A22, wherein the pumping section is a multipart member.
A24 Blood pump according to paragraph A23, wherein the main portion comprises a first main portion part and second main portion part.
A25 Blood pump according to paragraph A24, wherein the main portion comprises a third main portion part disposed within the first main portion part and/or within the second main portion part.
A26 Blood pump according to any one of the paragraphs A24 or A25, wherein the first main portion part is made of a metallic material, preferably of titanium or a titanium alloy
A27 Blood pump according to any one of the preceding paragraphs A24 to A26, wherein the second main portion part is made of a plastic material, preferably of polycarbonate.
A28 Blood pump according to any one of the preceding paragraphs A24 to A27, wherein the third main portion part is made of a plastic material, preferably of polycarbonate.
A29 Blood pump according to any one of the preceding paragraphs A1 to A28, wherein the blood flow outlet comprises a first spout.
A30 Blood pump according to paragraph A29, wherein the first spout extends tangentially to the longitudinal axis of the pump.
A31 Blood pump according to any one of the preceding paragraphs A24 to A30, wherein the blood flow outlet is provided on the second main portion part.
A32 Blood pump according to any one of the preceding paragraphs A29 to A31, wherein the blood pump further comprises a tubular graft connected to the first spout.
A33 Blood pump according to paragraph A32, wherein the blood pump further comprises a fixation member and a bushing with a circumferential undercut, wherein the bushing is disposed about the first spout, wherein the tubular graft is disposed about the bushing, and wherein the fixation member is configured to clamp the tubular graft to the circumferential undercut.
A34 Blood pump according to any one of the preceding paragraphs A32 or A33, wherein an exoskeleton is disposed about the tubular graft
A35 Blood pump according to paragraph A34, wherein the exoskeleton comprises a plurality of hingewise coupled first members, each first member preferably comprising an annular base member.
A36 Blood pump according to paragraph A35, wherein each of the first members comprises two attachment protrusions.
A37 Blood pump according to paragraph A36, wherein each of the attachment protrusions is provided on an axially extending lug, wherein the lugs preferably extend from the annular base member.
A38 Blood pump according to paragraph A37, wherein the lugs extend in a first direction and are provided exactly opposite to each other.
A39 Blood pump according to any one of the preceding paragraphs A35 to A38, wherein each of the first members comprises two attachment openings.
A40 Blood pump according to paragraph A39, wherein each of the attachment openings is provided on an axially extending lug, wherein the lugs preferably extend from the annular base member.
A41 Blood pump according to paragraph A40, wherein the lugs extend in a second direction and are provided exactly opposite to each other.
A42 Blood pump according to any one of the preceding paragraphs A35 to A41, wherein the exoskeleton comprises a second member hingewise coupled to the first member farthest from the pumping section.
A43 Blood pump according to paragraph A42, wherein a sleeve is coupled to the second member, the sleeve being disposed about the tubular graft.
A44 Blood pump according to paragraph A42 or A43, wherein the second member comprises an axial through hole, wherein the tubular graft extends through the axial through hole, wherein the diameter of the axial through hole at least partially reduces in a direction towards the pumping section.
A45 Blood pump according to any one of the preceding paragraphs A1 to A45, wherein a first sensor is disposed in proximity to the blood flow outlet.
A46 Blood pump according to paragraph A45, wherein the first sensor is disposed within the pumping section.
A47 Blood pump according to paragraph A45 or A46, wherein the first sensor is a first optical sensor.
A48 Blood pump according to any one of the preceding paragraphs A1 to A47, wherein a second sensor is disposed in proximity to the blood flow inlet.
A49 Blood pump according to any paragraph A48, wherein the second sensor is a second optical sensor.
A50 Blood pump according to any one of the preceding paragraphs A1 to A49, wherein a diameter of the main portion is larger than a diameter of the inlet portion.
A51 Blood pump according to paragraph A50, wherein the pumping section comprises a tapered portion connecting the main portion and the inlet portion.
A52 Blood pump according to any one of the preceding paragraphs A1 to A51, wherein the blood flow inlet comprises at least one radial inlet opening, preferably a plurality of radial inlet openings.
A53 Blood pump according to paragraph A52, wherein the plurality of radial inlet openings are evenly distributed around the circumference of the inlet portion.
A54 Blood pump according to any one of the preceding paragraphs A1 to A53, wherein the blood flow inlet comprises an axial inlet opening.
A55 Blood pump according to any one of the preceding paragraphs A1 to A54, wherein the inlet portion is integrally formed with at least a part of the main portion, preferably with the first main portion part.
A56 Blood pump according to any one of the preceding paragraphs A1 to A55, wherein the pump comprises a drive shaft driven by the motor, wherein the pump element is supported on the drive shaft.
A57 Blood pump according to any one of the preceding paragraphs A1 to A55, wherein the motor comprises a drive section comprising a stator and the pump element comprises magnets disposed adjacent to the electrically magnetized zones in the stator of the drive section.
A58 Blood pump according to any one of the preceding paragraphs A1 to A57, wherein the pumping section comprises a second spout.
A59 Blood pump according to paragraph A58, wherein the second spout extends tangentially to the longitudinal axis of the pump.
A60 Blood pump according to any one of the preceding claims A1 to A57, wherein the pumping section comprises a guide opening.
A61 Blood pump according to paragraph A60, wherein the guide opening extends tangentially to the longitudinal axis through the pumping section.
A62 Blood pump according to any one of the preceding paragraphs A56 to A61, wherein the second spout or the guide opening is configured to guide an elongated connection assembly.
A63 Blood pump according to paragraph A62, wherein the elongated connection assembly comprises one or more of a catheter, a motor cable, a first optical fiber, a second optical fiber and a purge fluid channel.
A64 Blood pump according to any one of the preceding paragraphs A1 to A63, wherein the main portion comprises a circumferential section with an increased surface roughness.
A65 Blood pump according to paragraph A64, wherein the circumferential section with the increased surface roughness is generated by material ablation.
A66 Blood pump according to paragraph A64 or A65, wherein the circumferential section with the increased surface roughness is generated by laser material ablation or by laser bead blasting.
A67 Blood pump according to any one of the preceding paragraphs A1 to A66, wherein the pumping section comprises a connective pathway which connects the blood flow inlet and the blood flow outlet, wherein preferably the connective pathway is partially formed by an annular gap.
B1 Blood pump assembly comprising a blood pump according to any one of the preceding paragraphs A1 to A67 and an expandable member, the expandable member being configured to be disposed within the inlet portion at a free axial end of the inlet portion, wherein the expandable member is preferably configured to be inflatable and deflatable.
B2 Blood pump assembly according to paragraph B1, wherein the expandable member comprises a base portion and a tip portion.
B3 Blood pump assembly according to paragraph B2, wherein the tip portion has a conical shape.
B4 Blood pump assembly according to paragraph B2 or B3, wherein the base portion is provided in the inlet portion when the expandable member is inflated.
B5 Blood pump assembly according to any one of the preceding paragraphs B2 to B4, wherein the tip portion extends from the axial inlet opening when the expandable member is inflated.
B6 Blood pump assembly according to any one of the preceding paragraphs B1 to B5, wherein the expandable member comprises a central opening for a guide wire to pass through.
B7 Blood pump assembly according to any one of the preceding paragraphs B1 to B6, wherein the expandable member is inflated with a liquid, preferably with a NaCl-solution.
C1 Method for placing a blood pump assembly according to any one of the preceding paragraphs B1 to B6 within a patient, the method comprising the following steps:
   puncturing the patient's heart; and
   placing at least a portion of the blood pump assembly through the puncture.
C2 Method according to paragraph C1, wherein the step of placing at least a portion of the blood pump assembly through the puncture comprises introducing a guide wire through the puncture.

C3 Method according to paragraph C1 or C2, wherein the step of placing at least a portion of the blood pump assembly through the puncture comprises dilatating the puncture.

C4 Method according to any one of the preceding paragraphs C1 to C3, wherein the step of placing at least a portion of the blood pump assembly through the puncture comprises inflating the expandable member.

C5 Method according to paragraph C4, wherein the step of placing at least a portion of the blood pump assembly through the puncture comprises advancing the blood pump along the guide wire.

C5 Method according to paragraph C5, wherein the step of placing at least a portion of the blood pump assembly through the puncture comprises moving the tip portion of the expandable member through the puncture.

C6 Method according to paragraph C6, wherein the step of placing at least a portion of the blood pump assembly through the puncture comprises deflating and removing the expandable member after the inlet portion is received within a space of the patient's heart.

The term “at least partially” as used herein denotes in total or only in parts. Terms like “first”, “second” or “third” do not denote a specific order, but are only intended to semantically differentiate between the elements.

### LIST OF REFERENCE NUMBERS

10 blood pump
12 pumping section
14 blood flow inlet
16 blood flow outlet
18 axial pump
20 motor
22 pump element/impeller
24 main portion
26 inlet portion
28 attachment portion
30 pump mounting portion
32 inner peripheral surface of main portion
34 magnetic circuit jacket
36 smaller diameter portion of magnetic circuit jacket
38 larger diameter portion of magnetic circuit jacket
40 tapered transition portion of magnetic circuit jacket
42 clamping portion
44 outer peripheral surface of main portion
46 slotted annular member of attachment portion
48 tangential fastener
50 first circumferential end of slotted annular member
52 second circumferential end of slotted annular member
54 slot
56 sealing element
58 first main portion part
60 second main portion part
61 third main portion part
62 first spout
64 tubular graft
66 fixation member
68 bushing
70 circumferential undercut of bushing
72 exoskeleton
76 second member of exoskeleton
78 sleeve
80 annular base member of first member
82 attachment opening of first member
84 attachment protrusion of first member
86 attachment protrusion of second member
88 axial through hole of second member
90 first sensor
92 second sensor
94 first optical fiber of first sensor
96 second optical fiber of second sensor
98 tapered portion
100 radial inlet opening
102 axial inlet opening
104 elongated connection assembly
106 guide opening
108 catheter
110 motor cable
112 circumferential section with increased surface roughness
114 cover
116 central mounting opening
118 annular surface
120 drive shaft
122 attachment flaps
124 lug
210 blood pump
212 pumping section
220 motor
222 pump element/impeller
224 drive section
226 magnets
228 second spout
230 first bearing
232 second bearing
300 blood pump assembly
302 expandable member
304 guidewire
306 inflation and deflation hose
308 base portion of expandable member
310 tip portion of expandable member
312 central opening of expandable member
AG annular gap
LA longitudinal axis

1. A blood pump comprising:
a pumping section having a blood flow inlet and a blood flow outlet; and
an axial pump having a longitudinal axis and at least partially being disposed within the pumping section, the axial pump having a motor and a pump element, the motor being configured to drive the pump element so that a flow is generated between the blood flow inlet and the blood flow outlet of the pumping section,
wherein the pumping section comprises a main portion and an inlet portion,
wherein the inlet portion is configured to be placed within a space of a patient's heart,
wherein the blood pump further comprises a radially extending attachment portion disposed about the main portion, and
wherein the attachment portion is configured to be attached to an outer surface of the patient's heart.

2. The blood pump according to claim 1, wherein the main portion comprises a pump mounting portion extending radially inwardly from an inner peripheral surface of the main portion, wherein the motor comprises a magnetic circuit jacket disposed in the pump mounting portion, or wherein the motor comprises a drive section disposed in the pump mounting portion and configured to drive the pump element in a contactless fashion.

3. The blood pump according to claim 1, wherein the main portion comprises an axially extending clamping portion on an outer peripheral surface, wherein the attachment portion is detachably mounted to the clamping portion so that the attachment portion is attachable at any position along an axial extension of the clamping portion.

4. The blood pump according to claim 1, wherein
the attachment portion comprises a slotted annular member and a tangential fastener, wherein the slotted annular member is disposed about the main portion and comprises a first circumferential end and a second circumferential end being spaced from each other so that a slot is formed between the first circumferential end and the second circumferential end, wherein the tangential fastener is configured to move the first circumferential end relative to the second circumferential end so that a size of the slot is variable.

5. The blood pump according to claim 1, wherein the pumping section is a multipart member, wherein the main portion comprises a first main portion part and second main portion part.

6. The blood pump according to claim 1, wherein
the blood flow outlet comprises a first spout extending tangentially to the longitudinal axis of the axial pump.

7. The blood pump according to claim 6, further comprising a tubular graft connected to the first spout.

8. The blood pump according to claim 7, further comprising a fixation member and a bushing with a circumferential undercut, wherein the bushing is disposed about the first spout, wherein the tubular graft is disposed about the bushing, and wherein the fixation member is configured to clamp the tubular graft to the circumferential undercut.

9. The blood pump according to claim 7, further comprising an exoskeleton disposed about the tubular graft, wherein the exoskeleton comprises a plurality of hingewise coupled first members.

10. The blood pump according to claim 9, wherein the exoskeleton further comprises a second member hingewise coupled to the first member farthest from the first spout, wherein the second member comprises an axial through hole, wherein the tubular graft extends through the axial through hole, wherein a diameter of the axial through hole at least partially reduces in a direction towards the first spout.

11. The blood pump according to claim 1, further comprising a first sensor disposed in proximity to the blood flow outlet within the pumping section, and/or a second sensor disposed in proximity to the blood flow inlet.

12. The blood pump according to claim 1, wherein a diameter of the main portion is larger than a diameter of the inlet portion, and wherein the pumping section comprises a tapered portion connecting the main portion and the inlet portion.

13. The blood pump according to claim 1, wherein the axial pump comprises a drive shaft driven by the motor, wherein the pump element is supported on the drive shaft, or wherein the motor comprises a drive section comprising a stator and the pump element comprises magnets disposed adjacent to electrically magnetized zones in the stator of the drive section.

14. The blood pump according to claim 1, wherein the pumping section comprises a second spout extending tangentially to the longitudinal axis of the axial pump or a guide opening extending tangentially to the longitudinal axis through the pumping section, wherein the second spout or the guide opening is configured to guide an elongated connection assembly.

15. A blood pump assembly comprising:
a blood pump according to claim 1; and
an expandable member configured to be disposed within the inlet portion at a free axial end of the inlet portion.
